(19) United States
(12) Patent Application Publication
(10) Pub. No.: US 2025/0127496 A1
(43) Pub. Date: Apr. 24, 2025

(54) Tonsillar Oropharyngeal Lavage Device For Early HPV Associated Oropharynheal Cancer Detection

(71) Applicant: Robert Kanas, Lakeville, MI (US)

(72) Inventor: Robert Kanas, Lakeville, MI (US)

(21) Appl. No.: 18/491,932

(22) Filed: Oct. 23, 2023

(51) Int. Cl. A61B 10/04; G16H 50/30

(52) U.S. Cl. CPC A61B 10/04; G16H 50/30; A61B 2010/045

(57) ABSTRACT

A lavage device for retrieval of tonsillar cryptic epithelial cells of the oropharynx for the early detection of HPV associated oropharyngeal cancer. The device consists of two specialized designed tips. The first special conical tip is designed and adapts to the convex surface of the palatine tonsils, while the second tip is used to reach the lingual tonsil at the base of the tongue. Each tip is connected to a base handle, dual lumen tubing and a water pump and vacuum pump on an opposite end thereof. The lavage liquid collected from either the palatine tonsil or lingual tonsil is placed in a sterile collection container and then passed through a dual filtering system to have the epithelial cells examined in both a cytological and molecular manner at the appropriate laboratory. The lavage liquid collected may help in the early detection of cancers associated with the HPV virus.

### BACKGROUND OF THE INVENTION

### 1. Field of the Invention

The present invention generally relates to medical devices and more specifically relates to an endoscopic device to retrieve diagnostic epithelial cells from the tonsillar crypts of both palatine and lingual tonsils where early HPV associated oropharyngeal cancer (OPC) is thought to originate.

### 2. Description of Related Art

Any discussion of the prior art throughout the specification should in no way be considered as an admission that such prior art is widely known or forms part of common general knowledge in the field.

Medical devices have been known for numerous years. Typically, medical devices vary greatly in configuration depending on what procedure that the medical equipment is utilized for. One such procedure that medical devices are utilized for is endoscopic procedures for diagnostic purposes. During an endoscopic procedure a technique to obtain diagnostic tissue samples is generally utilized to allow laboratory diagnosis of a patient's medical condition. Endoscopic procedures usually consist of imagery to visualize pathologic conditions of internal organs, which are then sampled by known biopsy techniques such as tissue forceps or brush biopsy technique. The palatine and lingual tonsils, which are viewed by endoscopy are usually within normal limits. OPC generally originates in the deeper crypts of the tonsils making surface imagery via traditional endoscopy procedures ineffective since viewing into the crypts is not obtainable with current endoscopy procedures. In addition, current tissue collection procedures with endoscopy techniques are limited to sampling the surfaces of the tonsils instead of the deeper tonsillar crypts which may be the actual anatomic location of early OPC. Furthermore, with OPC thought to originate in the inaccessible deeper crypts of the tonsils that traditional endoscopy procedures and brush biopsy techniques are generally unsuccessful in obtaining diagnostic tissue. Therefore, current sampling techniques may be ineffective for sampling the deeper tonsillar crypts. While the current endoscopic techniques of brush and forceps biopsy may be suitable for the particular purpose to which they are addressed, they are not as suitable for sampling of the deeper tonsillar crypts.

Therefore, there is a need in the art for an endoscopic device that substantially departs from the conventional concepts and designs of the prior art. Furthermore, there is a need in the art for an endoscopic device that provides an apparatus primarily developed for the purpose of retrieving diagnostic epithelial cells from the deeper tonsillar crypts for the purpose of early detection of Human Papillomavirus (HPV) associated oropharyngeal cancer (OPC). Furthermore, there also is a need in the art for an endoscopic device that is easy to manufacture and easy to use for the doctor performing the procedure. There is also a need in the art for an endoscopic device that is capable of use on palatine and lingual tonsils.

### SUMMARY OF THE INVENTION

In view of the foregoing disadvantages inherent in the known types of medical devices now present in the prior art for the purpose of tissue sampling of oropharyngeal tonsils, the present invention provides a new endoscopic device, wherein the same can be utilized for the tissue sampling of the deeper tonsillar cryptic epithelial cells. The general purpose of the present invention, which will be described subsequently in greater detail, is to provide a new endoscopic device that has many of the advantages of the medical devices mentioned heretofore and many novel features that result in a new endoscopic device which is not anticipated, rendered obvious, suggested, or even implied by any of the prior art medical devices, either alone or in any combination thereof.

One object of the present invention may be that it provides an endoscopic medical device that may overcome the shortcomings of the prior art.

Another object of the present invention may be to provide an endoscopic device to view and access tonsils for tissue sampling.

Still another object of the present invention may be to provide an endoscopic device that tissue samples the anatomic site of early OPC.

Still another object of the present invention may be to provide an endoscopic device that samples the deeper crypts of the OPC tonsils, both palatine and lingual.

Yet another object of the present invention may be to provide an endoscopic device that may invert the tonsillar crypts making them accessible to tissue sampling.

Still another object of the present invention may be to provide an endoscopic device that overcomes limitations of surface sampling via forceps biopsy or brush biopsy of the prior art.

Still another object of the present invention may be to provide dual filtering of a liquid biopsy, wherein one filter having a larger porosity for conventional cytology while the smaller porosity filter is used for molecular studies.

Still another object of the present invention may be to provide early cytological diagnosis and molecular testing for early premalignant and malignant HPV associated OPC.

According to the present invention, the foregoing and other objects and advantages are obtained by a novel design for an endoscopic device for early HPV associated OPC detection. The endoscopic device generally comprises two separate lavage tips, each of which may be attached to a control handle or handle base to collect cells from the crypts of either the palatine tonsils or lingual tonsils. One of the tips has a conical shape that is attached to the control handle, and is capable of delivering a pulsating water spray to the palatine tonsil while simultaneously inverting the tonsillar crypts by applying a constant vacuum to the tonsil. The handle base may be connected to a dual plastic intruded hose that creates a conduit for both supplying a sterile saline solution to the tonsil and a separate vacuum line to both invert the tonsillar crypts while simultaneously retrieving the liquid tissue sample into a sterile collection container or trap. A central unit may have both a small water pump and medical grade vacuum connected to the tip and the handle of the endoscopic device via the intruded hosing. The endoscopic device may also include a second vacuum line designed to retrieve liquid tissue samples from the collection container and routed through a dual filtering system under controlled vacuum pressure. The filtering system may consist of two separate filters of different porosity, one having greater porosity then then the other, in order to trap and collect the tonsillar epithelial cells for diagnostic purposes. A waste vial/container may also be used to collect the filtered waste liquid for disposal. It should further be noted that the endoscopic device may also include a second handle and tip styled after a curved McIntosh blade to collect lingual tonsillar epithelial cells, which generally are located at the base of the tongue. The curved tipped blade may have a ball tip that rests in the epiglottic vallecula that contains multiple, small pin size vacuum ports to collect fluid and cells in the vallecula as they are being washed from the lingual tonsil. The lavage spray is delivered through multiple small water spray openings along the inner curve surface of the curved McIntosh like blade that aligns with the lingual tonsil at the base of the tongue and may have a plastic edge that seals the water spray at the outer edges of the lingual tonsil. The water spray may irrigate the lingual tonsillar crypts and then the lavage liquid is routed through grooves designed between the irrigation ports along the lesser curve (underside) of the curved blade tip. This ensures the vacuum retrieval of the lavage liquid to the sterile collection container for diagnostic purposes. It should further be noted that both of the endoscopic devices include and are connected or in communication with a water pump, a vacuum, electronic controls, hosing, a sterile saline solution or bag, a collection container and a dual filtering system.

One advantage of the present invention may be that it provides an endoscopic medical device that may overcome the short comings of the prior art devices.

Still another advantage of the present invention may be that it provides an endoscopic medical device to view and access tonsils for tissue sampling.

Another advantage of the present invention may be that it provides an endoscopic device that samples tissue of the anatomic site of early OPC.

Still another advantage of the present invention may be that it provides an endoscopic device that samples the deeper crypts of the OP tonsils, both palatine and lingual.

Still a further advantage of the present invention may be that it provides an endoscopic device that inverts the tonsillar crypts making them accessible to tissue sampling.

Still another advantage of the present invention may be that it provides an endoscopic device that overcomes the limitations of surface sampling via forceps biopsy or brush biopsy of the prior art.

Still another advantage of the present invention may be that it provides a dual filtering of the liquid biopsy, such that a larger porosity for conventional cytology is used while a smaller porosity filter for molecular studies is also used in the same endoscopic device.

Yet another advantage of the present invention may be that it provides an early cytological diagnosis and molecular testing for early pre-malignant and malignant HPV associated OPC.

Yet another advantage of the present invention may be to provide an endoscopic device that is cost effective to make and easy to use in collecting diagnostic cells.

Other objects, features and advantages of the present invention will become apparent from the subsequent description, and the appended claims taken in conjunction with the accompanying drawings.

The more important features of the invention have been outlined above, rather broadly, in order that the detailed description thereof may be better understood and in order that the present contribution to the art may be better appreciated. There are additional features of the invention that will be described hereafter and that will also help in forming the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows a plan view of an endoscopic device according to the present invention.

FIG. 2 shows a side view of a curved blade tip and handle according to the present invention.

FIG. 3 shows an underside view of a curved blade tip and handle according to the present invention.

FIG. 4 shows a top view of a curved blade tip and handle according to the present invention.

FIG. 5 shows a closeup of the irrigation ports and evacuation ports of the curved blade tip and handle according to the present invention.

FIG. 6 shows an internal view of vacuum channels of the curved blade tip and handle according to the present invention.

FIG. 7 shows a side view of the internal membrane/diaphragm of the curved blade tip and handle according to the present invention.

FIG. 8 shows an internal view of irrigation spray channels and parts of the curved blade tip and handle according to the present invention.

FIG. 9 shows a cross sectional line through the curved blade tip and handle according to the present invention.

FIG. 10 shows an enlarged cross sectional view of the curved blade tip and handle according to the present invention.

FIG. 11 shows a cross sectional view of the vacuum channels and parts of the curved blade tip according to the present invention.

FIG. 12 shows a cross sectional view of the irrigation ports and vacuum channels of the curved blade tip according to the present invention.

FIG. 13 shows a cross sectional view of the curved blade tip and handle according to the present invention.

FIG. 14 shows a side view of the conical tip and handle according to the present invention.

FIG. 15 shows a top view of the conical tip and handle according to the present invention.

FIG. 16 shows a plan view of a conical tip and handle according to the present invention.

FIG. 17 shows a cross sectional view of the conical tip and handle according to the present invention.

FIG. 18 shows an enlarged cross sectional end view of the conical tip and handle according to the present invention.

FIG. 19 shows a plan view of the dual lumen for use according to the present invention.

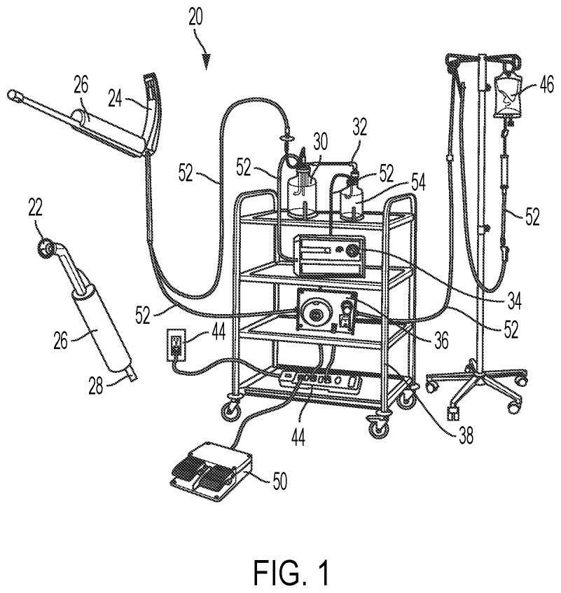

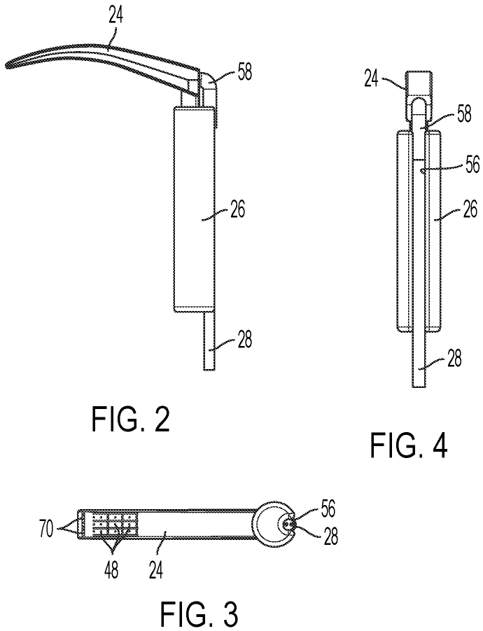

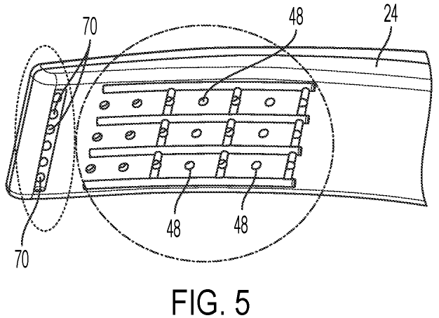

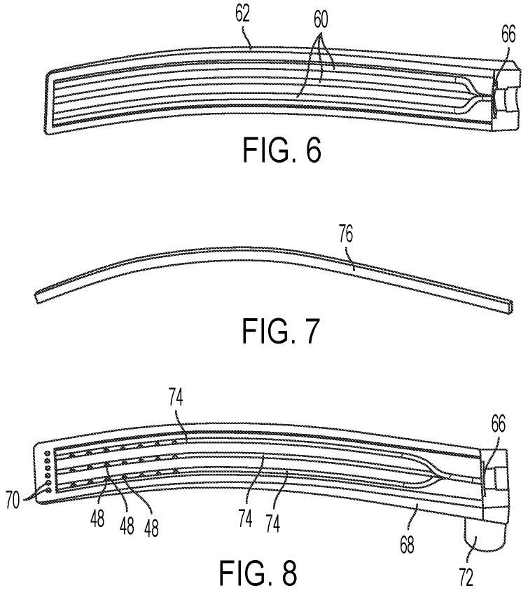

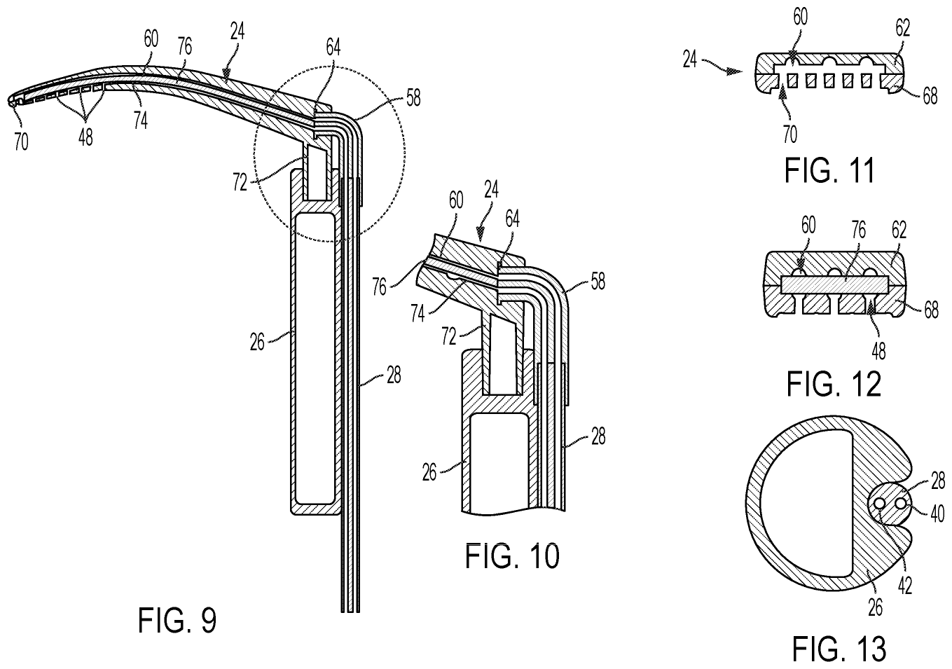

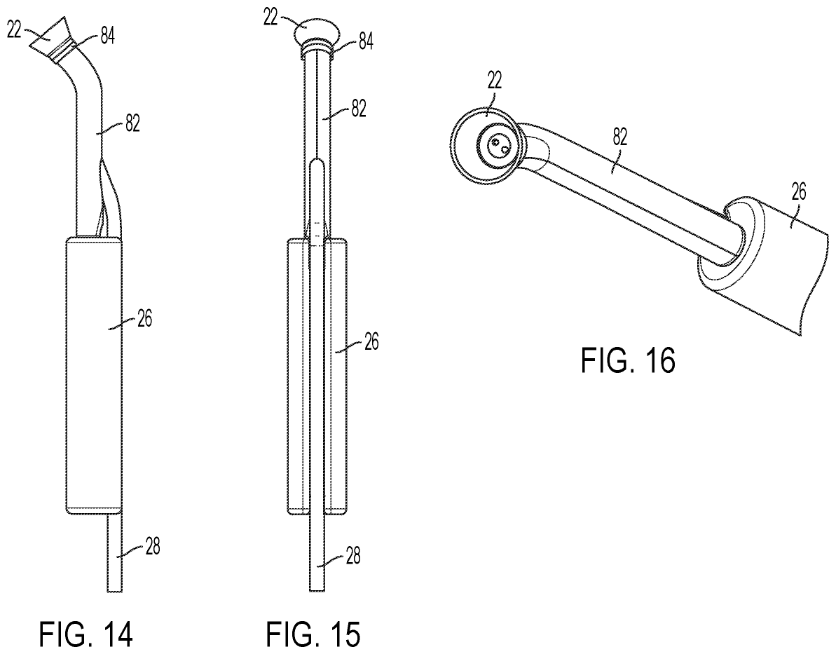

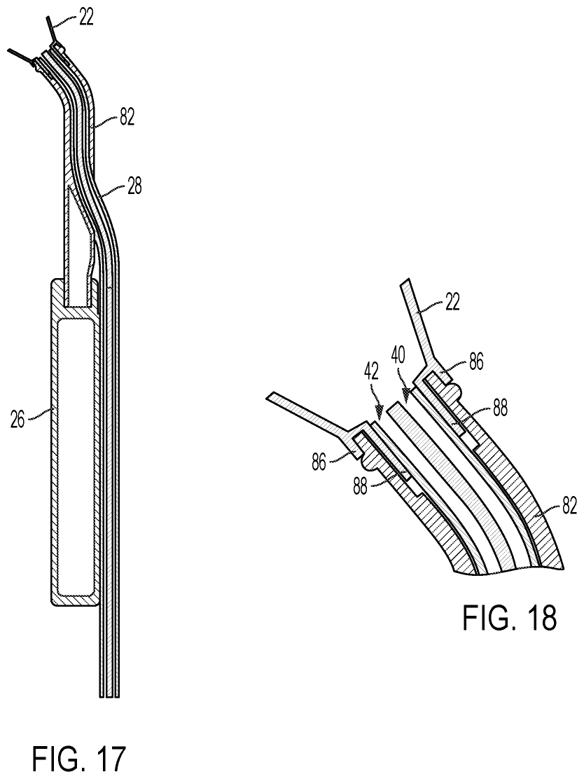

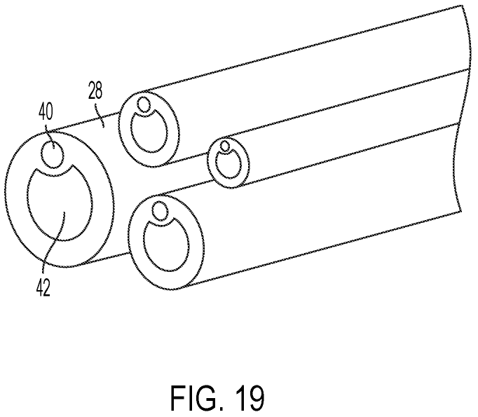

### DESCRIPTION OF THE EMBODIMENT(S)

Referring to the drawings, an endoscopic device 20 for early HPV associated oropharyngeal cancer detection is disclosed. The endoscopic device 20 of the present invention may be used as a lavage instrument for retrieval of tonsillar cryptic epithelial cells of the oropharyngeal for the early detection of human papillomavirus (HPV) associated oropharyngeal cancer (OPC). The endoscopic device 20 generally consists of two specialized design tips, one for the palatine tonsil which has a conical tip 22 and the other for the lingual tonsil, which has a curved blade tip 24. The first tip or the conical tip 22 is designed and adapted to be used on the convex surface of the palatine tonsils. The internal portion of the conical tip 22 has a special shower like water spray unit that delivers pulsating sterile saline to the palatine tonsil while a constant vacuum may also be applied through another orifice within the internal conical tip 22 causing inversion of the tonsillar crypts, thus exposing the crypts to the water spray and the expected retrieval of the epithelial cells within the deeper crypts to detect for early premalignant or malignant changes to the palatine tonsil. In addition to the conical tip 22, the endoscopic device 20 may also consist of a handle 26 which may be a sonic welded handle 26 which maybe sonically welded to the conical tip 22, an extruded dual tubing or dual lumen tubing 28 connecting the conical tip 22 and extending through the handle 26 and then ending in a sterile collection container 30. A filter apparatus 32 may be arranged in or near the sterile collection container 30 that consists of a double filter system to collect cells for both cytology and molecular studies. Furthermore, a central unit that houses the electronics to control both the vacuum pump 34 and water pump 36 necessary to operate the endoscopic device 20 of the present invention may be arranged on a cart 38, desk or other devices. It should further be noted that the sterile specimen container trap 30 may be placed between the conical tip 22 and central vacuum 34 to collect the liquid lavage sample from the tonsillar crypts. The lavage sample may be separately double filtered after collection with special, micro-sized filtering pores to trap the tonsillar epithelial cells. It should be noted that the filtering system 32 may be housed separately and also use a vacuum for filtering purposes. The central unit that houses both the water spray pump and vacuum may be connected to the lavage handle 26 with extruded medical grade hosing 52 that has at least a first vacuum channel 42 and a second irrigation spray 40 channel arranged therein wherein it directs the lavage sample to the sterile collection container 30. Furthermore, it should be noted that an electrical outlet or strip 44 and a sterile saline bag 46 may be required to support the endoscopic device 20 and to ensure correct usage of all components of the endoscopic device 20.

The second tip, also known as a curved blade tip 24, may also be used with the endoscopic device 20 of the present invention. It should be noted that the curved blade tip 24 replaces or is interchanged with the conical tip 22 which is used for the palatine tonsils, whereas as the curved blade tip 24 may be used to reach the lingual tonsils at the base of the tongue. The curved blade tip 24 is generally styled after a curved McIntosh blade that is well known in the medical art. The curved blade tip 24 generally uses a McIntosh blade that has been modified such that the lingual tonsil or curved blade tip 24 is engineered to have a suction tip and water spray nozzle near an end thereof to collect lingual (base of tongue) tonsillar epithelial cells. The curved blade tip 24 may include a ball tip that rests in the epiglottic vallecula that contain multiple, small pin size ports to collect fluid and cells that are irrigated from the lingual tonsil and channeled or directed to the vallecula. The lavage spray of the curved blade tip 24 may be delivered through multiple small water spray jet ports 48 along an inner surface curve of the curved blade tip 24 that aligns with the lingual tonsil at the base of the tongue. The spray tip may deliver a constant spray of a sterile saline to the tonsillar crypts while simultaneously being vacuumed from the modified ball tip that contain vacuum parts 70 from the vallecula into a sterile container 30 to be double filtered as described above. It should further be noted that the central unit system for generation and collection of the lavage system is the same as that described above for the conical tip 22 such that the same central unit system is shared for both the palatine and lingual tonsils and the tips 22, 24 associated therewith and used to get the cells necessary for studying or biopsy thereof.

The endoscopic device 20 of the present invention may include either a conical shaped tip 22 arranged on a handle 26, or a curved blade tip 24 arranged on a handle 26 for the generation and collection of the lavage liquid from either a palatine tonsil or lingual tonsil for diagnostic purposes. The endoscopic device 20 may include a central unit system that is used with either the conical tip 22 or the curved blade tip 24 according to the present invention. This central unit system may include the following apparatuses: a water pump 36, a vacuum 34, an electronic control 50, hosing 52, a sterile saline bag 46, a collection container 30, and a dual filtering system 32. It should be noted that the water pump 36, vacuum 34, collection container 30, hosing 52 and filters 32 generally are all commercially available in any medical supply setting. In one embodiment shown in the drawings, the central unit system may include a cart 38 or other container that holds a medical grade vacuum 34 and a medical grade water pump 36 in a predetermined area. The central unit system may also include a collection container 30 arranged on the cart 38 along with the necessary electrical outlets 44, either arranged on the cart or the cart is arranged near an electrical outlet in order to plug in all of the components to an electrical power source. Furthermore, a sterile saline bag 46 or other methodology of delivering sterile saline to either tip of the endoscopic device 20 is also used. In one embodiment shown, a rolling hanging system for supporting and holding a sterile saline bag 46 may be used, however any other methodology of securing a saline bag 46 at or near the endoscopic device 20 being used is also capable of being used according to the present invention. The central unit system may also include a foot pedal that electronically controls the operation of the endoscopic device tip 22, 24, such as the flow of the saline solution to the endoscopic tip and the vacuum being placed on the endoscopic tip for removing the lavage liquid to the collection container 30. In one embodiment shown, the collection container 30 may be a glass collection jar, however any other type of collection container 30 made of any other material may also be used. Arranged in proximity to the collection container 30 may be a waste container 54 for capturing and storing left over lavage liquid for proper disposal of the medical waste. It should be noted that the collection container 30 is a sterile device that may include a dual filtering system 32 arranged near or in the collection container 30, wherein one of the filters may have a larger porosity than the other filter, which has a smaller porosity, thus allowing for further collection of tonsillar epithelial cells for diagnostic purposes. It should be noted that any type of medical grade hosing 52 may be used to interconnect the various portions of the central unit system to one another and eventually to the end of the endoscopic device tips 22, 24. The sterile saline bag 46 may be connected via any known medical grade hosing or tubing 52 to the water pump 36. In one contemplated embodiment, the sterile bag 46 of saline may be fed by gravity to the water pump 36 and then from the water pump 36 via another medical grade hose 52 to the conical tip 22 or curved blade tip 24 of the endoscopic device 20. This may allow for transfer of the sterile saline through the water pump 36 to the tip 22, 24 of the endoscopic device 20 for spraying onto to either of the tonsils.

Another component of the central unit system is the vacuum pump 34 that may control the level of vacuum delivered to the tonsils 34. The sterile collection container 30 may be arranged between the handle base 26 and the central vacuum 34. A first piece of medical grade tubing 52 is arranged between the handle base 26 of the endoscopic device 20 and a sterile collection container 30. A second piece of medical tubing 52 is arranged between the sterile collection container 30 and the vacuum pump 34. The vacuum pump 34 may then create a vacuum through the sterile container 30 to the tip 22, 24 of the endoscopic device 20 thus allowing for any lavage liquid collected through, either the conical tip 22 or the curved blade tip 24, to be trapped in the sterile collection container 30 and then routed through the dual filtering system 32 of the endoscopic device 20. After the lavage liquid is routed through the dual filtering system 32, the remaining liquid may be transferred to a waste container 54 that is connected via medical grade hosing 52 to the sterile container 30 after the dual filtering system 32. It should further be noted that the waste container 54 may also include a medical grade hose 52 connected to the vacuum pump 34 to ensure proper movement of any waste into the waste container 54. It should be noted that the dual filtering system 32 may have different porosities, thus having larger pores in one of the filters and smaller pores in the other filter in order to separate the epithelial cells collected by the filtering system 32 for both cytology and molecular studies. All of the central unit system components maybe mobile or may be fixed into a certain position in order to be used for testing of the tonsils for cancer cells and the like. Therefore, the waste container 54 may also be connected via hosing 52 to the vacuum 34 and thus creating a vacuum that may move the lavage liquid arranged in the sterile container 30 through the filters 32 which is arranged therebetween. Any of the epithelial cells collected maybe used for cytological diagnosis and molecular diagnostic for early cancer or the like.

The curved blade tip or McIntosh like blade tip 24 of the endoscopic device 20 generally includes a handle base 26 that has a cylindrical shape. The handle base 26 may be made of any rigid material, such as any hard plastic, ceramic, composite, metal, natural material, or manmade material. Generally, the handle 26 may have any predetermined length and diameter depending on the design requirements of the endoscopic device 20. In one contemplated embodiment, the handle base 26 may have an outer diameter of approximately one inch and a length of approximately five inches. However, any other range from a quarter inch diameter up to three inches in diameter and a length from three inches up to twenty inches may also be used. The handle 26 may have a generally circular channel or track 56 running along an entire outside edge thereof. Arranged within the circular channel 56 may be a piece of dual lumen tubing 28. In one contemplated embodiment, dual lumen tubing 28 may have a predetermined outer diameter and an inner bore having a first vacuum 42 and second irrigation spray 40 generally circular orifices arranged therein. One of the orifices 42 may have a larger diameter than the other of the orifices 40, wherein the smaller diameter orifice 40 may be used to transfer the sterile saline solution or water to the endoscopic device tip 24 while the larger diameter orifice 42 may be used for the vacuum and thus removing the lavage liquid to the sterile collection container 30. In one contemplated embodiment, the larger inner diameter 42 may be approximately one eighth of an inch while the smaller inner diameter 40 for the second orifice may be one sixteenth of an inch, while the outside diameter of the double dual lumen tubing 28 may be approximately one quarter inch in diameter. However, any other size may be used for the diameters of the dual lumen tubing 28 arranged within and secured via an interference fit with a circular channel 56 arranged along one edge of the base handle 26. The curved blade tip 24 also includes a connector member 58 generally having an angled curve arranged near one end thereof. The connector member 58 may be arranged over one end of the dual lumen tubing 28 on one end thereof and arranged within the curved blade tip 24 on the other end thereof. The connector member 58 may have a first vacuum and second irrigation spray orifice arranged through an inner bore thereof, wherein one of the orifice aligns with the orifice 40 of the dual lumen tubing 28, whereas the second orifice aligns with second orifice 42 of the dual lumen tubing 28, such that one of the orifices may be used to send saline solution to the end of the curved tip blade 24, while the other orifice may be used to create a vacuum or suction through the bottom portion of the curved tip blade 24. It should be noted that the dual lumen tubing 28 may be connected via any connector or manifold to the saline solution via the water pump 36 and to the sterile collection container 30 via the vacuum pump 34. It should be noted that the medical tubing 52 may be arranged within a manifold or valve component that may direct the saline solution into the smaller orifice 40 of the dual lumen tubing 28 while the vacuum tubing 52 may be connected and in communication with the larger of the dual lumen tubing orifice 42. It should be noted that it is also contemplated that the dual lumen tubing 28 may run or extend directly from the handle 26 of the endoscopic device 20 to the curved blade tip 24. The curved blade tip 24 may be a two piece construction. The top portion 62 of the curved blade tip 24 may include a plurality of channels or tracks 60 arranged in an inner surface thereof that may deliver constant vacuum to the ball tip of the curved blade tip 24. It should be noted that in one contemplated embodiment a main branch channel 60 may be divided off into three separate channels 60 running along a predetermined length or generally the entire length of the top portion 62 of the curved blade tip 24. The connector component 58 arranged between the dual lumen tubing 28 and one end of the curved blade tip 24 includes a circular flange or lip 64 arranged at or near one end thereof, wherein the lip 64 may be arranged within a circular orifice, slot or cavity 66 arranged in or near an end of the top portion 62 and bottom portion 68 of the curved blade tip 24. The circular flange 64 may be arranged within the circular cavity 66 of the curved blade tip 24 thus holding and securing the generally circular connector member 58 to the curved blade tip 24 in a predetermined position. It should be further noted that the opposite end of the connector member 58 may be arranged over the outer diameter of the dual lumen tubing 28 and is secured thereto via an interference fit of the like. However, any other mechanical or chemical fastening technique may also be used to secure the end of the dual lumen tubing 28 within the end of the connector member 58. Therefore, the top portion 62 of the curved blade tip 24 generally has a rectangular shape with a predetermined curve occurring over the length thereof. Thus, when viewed from a side it appears to have a wing like shape. The vacuum channels 60 arranged within the bottom surface of the top portion 62 of the curved blade tip 24 extend from a predetermined distance from the end of the top portion 62 of the tip 24 to the circular cavity 66 where the connector member 58 is arranged on the opposite end of the curved blade tip 24. It should be noted that the channels 60 have a predetermined depth and are capable of delivering constant vacuum to a plurality of orifices or ports 70 are arranged near an end of the bottom portion 62 of the curved blade tip 24. In one contemplated embodiment at least six orifices or ports 70 are arranged near the tip for use in vacuuming the irrigation spray from the lingual tonsils during removal of epithelial cells therefrom. The end of the curved blade tip 24 may have a generally circular or ball like end with six vacuum ports 70 to retrieve the lavage sample.

The curved bladed tip 24 also includes a bottom portion 68. The curved blade tip bottom portion 68 includes a circular conical post 72 extending from a first end thereof. The conical post 72 may be solid or hollow depending on the design requirements. The conical post or rod 72 may be arranged via a press fit interengagement into a circular orifice arranged in an end of the handle base 28. In one contemplated embodiment, the circular post 72 may have a taper like shape that interacts with a tapered like shape in a generally circular orifice of the handle member 28 to ensure an interengagement and secure press fit interference connection between the bottom portion of the blade tip 24 and the base handle 28. It should also be known that any other known chemical fastening or mechanical fastening technique may be used to secure the curved blade tip 24 into the top of the base handle 28 including but not limited to sonic welding, glue, epoxy, adhesives, etc. The bottom portion 68 of the curved blade tip 24 also generally has a rectangular shape with a curved shape when viewed from a side such that it also resembles a wing. The bottom portion 68 also includes a circular cavity or slot 66 that is arranged directly across from the circular cavity 66 on the top portion 62 thereof, in order to hold and secure the circular flange 64 of the connector member 58 therein. The bottom portion 68 also includes a plurality of irrigation spray channels or tracks 74 arranged therein, wherein the channels 74 extend from the circular cavity 66 to a predetermined distance from the end of the curved blade tip 24. The channel 74 begins as a single channel at the circular cavity 66 and then branches off into a first, second and third irrigation spray channel 74, just as the top portion 62 of the curved blade tip 24. The first, second and third channel 74 of the bottom portion 68 of the curved blade tip 24 may include a plurality of orifices 48 arranged near or adjacent to an end thereof. These orifices 48 may allow for a constant water irrigation spray of the lingual tonsil to occur while the curved blade tip 24 is arranged on the back of the tongue during the biopsy procedure. Thus, the lavage liquid is delivered through the spray irrigation ports 48 and then sucked or drawn through the orifices 70 arranged within the three channels 60 of the top portion 62 of the curved blade tip 24 and directed back through the dual lumen tube 28 to the sterile collection container 30 via the vacuum pump 34 of the endoscopic device 20. It should be known that circular orifices 48 generally are used, however any other shaped orifice may be used through the bottom surface of the curved blade tip 24. Thus, the curved blade tip 24 includes a plurality of evacuation ports or orifices 70 arranged on a top surface thereof and a plurality of irrigation ports or orifices 48 arranged on a bottom surface thereof, wherein the evacuation vacuum ports 70 are arranged near an end of the ball like curved end of the curved blade tip 24 while the irrigation spray ports 48 are arranged a predetermined distance from that end and from the evacuation or vacuum ports 70 on the top surface thereof and generally in alignment with water channels 74 arranged in an inner top surface of the bottom portion 68 of the curved blade tip 24. It should also be noted that it is also contemplated that a plurality of channels may be arranged on the outer bottom surface of the curved blade tip 24, where the irrigation ports 48 are arranged in a channel and some of the evacuation ports 70 are also arranged in channels depending on the design requirements. One channel may only be arranged directly over or on the irrigation ports 48 in the bottom surface of the blade tip 24 while some, but not all of the evacuation ports 70, may be arranged in channels in the top surface of the curved blade tip 24. These channels may be used in directing the lavage liquid into the evacuation ports 70 for eventual transportation to the sterile collection container 30. Any sized orifice may be used for either the irrigation ports 48 or the evacuation ports 48 of the curved blade tip 24. The curved blade tip 24 also may include an internal membrane or diaphragm 76 arranged between a bottom surface of the top portion 62 and a top surface of the bottom portion 68 of the curved blade tip 24. In one contemplated embodiment, the internal diaphragm 76 generally may have a rectangular shape that is less than the entire length of the curved blade tip 24 and has a predetermined thickness and whereas the width is generally smaller than that of the curved blade tip 24. In one contemplated embodiment the internal diaphragm 76 is made of a silicone material, however any other soft plastic, rubber, composite, man-made material or natural material may be used as long it is capable of being waterproofed and not allowing water to pass from the bottom portion 62 of the curved blade tip 24 into the top portion 68 of the curved blade tip 24. The width of the internal diaphragm 76 is such that it sits within a notch arranged within the bottom portion 68 of the curved blade tip 24 and a notch in the top portion 62 of the curved blade tip 24. The internal silicone diaphragm 76 may be used to keep the water channels 74 of the bottom portion 68 of the curved blade tip 24 and the vacuum channels 60 of the top portion 62 of the curved blade tip 24 from interacting with one another in any manner. This may ensure that the saline solution passing through the bottom portion 68 of the curved blade tip 24 and out the irrigation ports 48 at the underside of the curved blade tip 24 and the lavage liquid being drawn into the top portion 62 of the curved blade tip 24 do not interact with one another and contaminate the sample being collected from the lingual tonsils of the tongue. The internal diaphragm 76 may mimic the outer shape of the generally rectangular notch or cutout of the bottom portion and the top portion of the curved blade tip 24. A predetermined portion of the internal diaphragm 76 may sit within the notch formed in the top portion 62 of the curved blade tip 24 and a predetermined portion may sit within the notch of the bottom portion 68 of the curved blade tip 24 according to the present invention. It should be noted that the top portion 62 and bottom portion 68 of the curved blade tip 24 may be connected to each other via either a sonic welding or any other type of mechanical or chemical fastening technique, such as plastic welding, epoxy, glue, or any other method of connecting a plastic material to another plastic material. Therefore, it should further be noted that any type of hard plastic may be used for the curved shape tip 24, the handle base 26 and the connector member 58 of the curved shaped tip 24 of the endoscopic device 20 according to the present invention. It should also be noted that any size may be used other than the sizes described above. Generally, the length of the curved blade tip 24 including the connector member 58 is approximately five inches. However, any other shape may be used other than those described herein, such that the curved blade tip 24 including the connector member 58 may be anywhere from one inch up to twenty four inches, depending on the use and/or the person or animal the system is being used thereon.

A second tip to sample the palatine tonsils that may be used with the endoscopic device 20 of the present invention is generally a conical tip 22. It should be noted that the handle base 26 may also be hollow or solid depending on the design requirements. The base handle 26 of the conical tip 22 is the same as that of the curved blade tip 24. It will not be discussed in further detail herein, see above for details. Furthermore, the dual lumen tubing 28 is also the same as that of the curved blade tip 24 and the connection of the dual lumen tubing 28 to the medical tubing 52 from the water pump 36 and from the vacuum pump 34 also would be the same. It should be noted that the dual lumen tubing 28 is arranged within the same circular orifice or channel 56 arranged along one side of the base handle 26 of the conical tip 22. In the conical tip 22 for the endoscopic device 20, the dual lumen tubing 28 extends to an end of the sheath member 82. Generally, the sheath or sheath member 82 of the conical tip 22 is arranged within a circular orifice on one end of the base handle 26. The sheath 82 generally has a cylindrical shape and a first end is arranged and secured within the base handle 26 circular orifice via either a press fit and/or other methodology to secure the end of the sheath 82 within the end of the base handle 26. It should be noted that any known medical, mechanical or chemical fastening technique may be used, such as welding, interference fit, snap fit, gluing, epoxy, etc., as long as the handle 26 is removably secured or permanently secured to the end of the handle 26, it may work with the present invention. Therefore, it should be noted that the same base handle 26 may be used for either the conical tip 22 or the curved blade tip 24 and may be secured thereto via a press fit, a snap or other mechanical or chemical connection to the end of the handle 26. It should be noted that the base handle 26 of the curved blade tip 24 also may have a portion of the connector member 58 arranged within the circular channel 56 of the base handle 26. Therefore, the sheath 82 of the conical tip 22 may be either permanently or removably secured within the base handle end. The same is true for the curved blade tip 24. The sheath 82 also includes a generally circular inner bore arranged through a predetermined portion thereof. The bore generally extends a predetermined distance from a second end of the sheath 82 and exits through a predetermined side surface of the sheath 82 at a predetermined distance from the second end. The dual lumen tubing 28 is arranged through and within the bore such that an end of the dual lumen tubing 28 aligns with or at a generally same plane as the second end of the sheath 82. The dual lumen tubing 28 thus extends from the second end of the sheath 82 through an inner bore and out a surface of the sheath 82. The sheath 82 has a generally oval like shaped orifice through a surface thereof, wherein the dual lumen tubing 28 may pass through this orifice until it is arranged at or near the end of the sheath 82. It should be noted that the sheath 82 also may have a curve arranged near a top edge thereof, thus allowing for easier access to the palatine tonsils by the conical tip 22. The sheath 82 may also include a circumferential lip 84 extending from the second end thereof or a predetermined distance below the second end thereof. The circumferential lip 84 may be used to interengage with a flange of the conical tip member 22 secured to the end of the sheath 82 of the conical tip 22. It should be noted that the conical tip 22 generally has a conical shape when viewed in cross section. Thus, it appears to have a cone or cup like shape when viewed in cross section. The conical tip 22 also includes a first flange 86 and a second flange 88 extending from a bottom portion thereof. The first flange 86 contacts and interengages with the circumferential lip 84 extending from an outside surface of the sheath 82, while the second flange 88 is radially internal of the first flange 86 and extends a predetermined distance from the bottom of the conical tip 22. The second flange 88 extends a further distance than the first flange of the conical tip 22. The sheath 82 also includes a circular cavity arranged within the second end of the sheath 82. This circular cavity may receive and hold the second flange 88 extending from a bottom surface of the conical tip 22. Thus, the second end of the sheath 82 may be sandwiched between the first flange 86 and the second flange 88 of the conical tip 22, such that the conical tip 22 is held securely in place via either an interference fit, a snap fit, or any other mechanical or chemical fastening technique, such as glue or epoxy. This may ensure that the conical tip 22 is secured to the second end of the sheath 82. It should further be noted that the dual lumen tubing 28 outer diameter or outer surface thereof may interact with and be secured in proper position to the end of the sheath 82 via an inner surface of the second flange 88 of the conical tip 22. The second flange 88 of the conical tip 22 has an outer surface that engages and contacts an inner surface of the sheath 82. The first flange 86 of the conical tip 22 has an inner surface that is engaged with and contacts an outer surface of the sheath member and an end of the first flange 86 of the conical tip 22 is engaged with a portion of the circumferential lip arranged around an outer surface of the sheath 82. Thus, the saline solution may spray through the smaller diameter orifice 40 of the dual lumen tubing 28 while the larger diameter orifice 42 may create suction and suck the lavage liquid after being sprayed on the palatine tonsil into the sterile collection container 30 and then through the dual filtering system 32 and into a waste container 54. It should be noted that all components of the conical tip 22 or curved blade tip 24 are made of a hard plastic material or any other plastic, metal, ceramic, composite, man-made or naturally occurring material. It should be noted that in one contemplated embodiment the length from the bottom of the base handle 26 to the top of the conical tip 22 is approximately eight and a half inches for the conical tip 22 endoscopic device 20. However, any other length and diameter of any of the tubing, base handle, etc., may be used. In fact, it is contemplated that anywhere from two inches to forty eight inches may be used for the conical tip endoscopic device 20.

Therefore, the endoscopic device 20 of the present invention is capable of having two separate lavage tips attached to a control handle 26 to collect cells from the crypts of either the palatine tonsils or lingual tonsils. First, a conical shape tip 22 may be attached to the rigid base handle 26, similar to a handle of a rigid laryngoscope, with the capacity to deliver a pulsating water spray to the palatine tonsil while simultaneously inverting the tonsillar crypts by applying a constant vacuum to the tonsil. The handle base 26 may be connected to a dual lumen plastic intruded hosing 28 that create a conduit for both supplying a sterile saline solution to the tonsil and a separate vacuum line to both invert the tonsillar crypts while simultaneously retrieving the liquid tissue sample into a sterile collection container 30. The intruded dual lumen hosing 28 is connected and regulated by a central unit system that uses both a small water pump 36 and a medical grade vacuum 34. The water pump 36 may be supplied with a sterile bag of saline 46 fed by gravity or any other methodology to the pump 36. It should be noted that the vacuum pump 34 may control the level of vacuum delivered to the tonsils and a sterile collection container 30 may be situated between the handle base 26 and central vacuum 34. Furthermore, a second vacuum line may be designed to retrieve the liquid tissue sample from the collection container 30 and routed through a dual filtering system 32 under controlled vacuum pressure. This filtering system 32 may consist of two separate filters of different porosities, such that one has a greater porosity than the other and one may trap and collect the tonsillar epithelial cells for diagnostic purpose, both to cytological and molecular testing. It should be noted that each filter may be retrieved from their respective holding vessels and subsequently placed in the appropriate fixative container for transportation to a diagnostic laboratory. Furthermore, the waste vial or container 54 that collects the filtered waste liquid for disposal is then properly disposed of according to all medical guidelines. Furthermore, if the lingual tonsils are to be biopsied, a second handle or curved blade tip 24 may be used with the base handle 26 of the present invention. This curved blade tip 24 may be a generally modified McIntosh blade that may have a suction tip and a water spray nozzle to collect lingual base of tongue or tonsillar epithelial cells. The curved blade tip 24 generally has a ball tip that rests in the epiglottic vallecula that contain multiple, small pin size ports to collect fluid and cells in the vallecula as they are being washed from the lingual tonsil. The lavage spray is delivered through multiple small water spray openings along the inner curve of the McIntosh blade that aligns with the lingual tonsil at the base of the tongue and has a plastic edge that seals the lavage spray at the outer edges of the lingual tonsil. The water spray then irrigates the lingual tonsil crypts and the lavage liquid is channeled within grooves designed between the irrigation ports 48 along the lesser curve (under-side of curved blade tip) to route the liquid lavage to the vallecula for vacuum retrieval to the sterile collection container 30 and subsequent filtering as described above for transportation to the laboratory for diagnostic purposes. It should be noted that the apparatus used in conjunction with the endoscopic device of the present invention for the generation and collection of lavage liquid for diagnostic purposes includes, but is not limited to, a water pump 36, vacuum 34, electronic controls 50, hosing 52, sterile saline bag 46, collection container 30, and subsequent dual filtering system 32 are all shared and are in common for each of the palatine and lingual tonsil tips 22, 24. It should further be known that the water pump 36, vacuum source 34, collection container 30, hosing 52 and filters 32 are all commercially available and are well known in the art.

This description is for illustrative purposes only and should not be construed to limit the present invention in any way. Thus, a person skilled in the art will appreciate that various modifications might be made to the present and disclosed embodiments without departing from the scope and spirit of the present invention, which is defined in terms of the claims below. Other aspects, features and advantages may be apparent upon an examination of the attached drawings and the appended claims.

What is claimed is:

1. An endoscopic device for early HPV associated oropharyngeal cancer detection, said device comprising:
a water pump;
a saline solution connected to said water pump with a first medical grade hose;
a handle having a predetermined tip arranged therein, said handle connected to said water pump with a second medical grade hose;
a vacuum component connected to a first collection container via a third medical grade hose;
a fourth medical grade hose arranged between said handle and said first collection container;
a second collection container connected to said first collection container via a fifth medical grade hose; and
a filter system arranged between said first collection container and said second collection container.

2. The device of claim 1 wherein said handle having a track arranged therein.

3. The device of claim 2 wherein the track having a dual lumen tube arranged therein.

4. The device of claim 3 wherein a predetermined portion of said dual lumen tube is arranged within said track.

5. The device of claim 4 wherein said predetermined tip is a palatine tip for use on a palatine tonsil.

6. The device of claim 5 wherein said palatine tip having a generally conical shape.

7. The device of claim 6 wherein said palatine tip having an orifice at a mid-point thereof.

8. The device of claim 7 further comprising a first flange and a second flange extending from a bottom of said palatine tip.

9. The device of claim 8 wherein said dual lumen tube having an irrigation port and an evacuation port arranged therethrough.

10. The device of claim 9 wherein said sheath covers a portion of said dual lumen tube.

11. The device of claim 10 further comprising a circumferential lip extending from an outer surface of said sheath.

12. The device of claim 11 wherein said palatine tip engages with and contacts surfaces of said sheath and engages with and contacts a surface of said dual lumen tube.

13. The device of claim 4 wherein said predetermined tip having a generally curved blade shape.

14. The device of claim 13 wherein said curved blade tip having a mounting rod extending from a bottom surface thereof.

15. The device of claim 14 wherein said curved blade having a plurality of evacuation ports and tracks at or near an end of said bottom surface of said curved blade tip, a plurality of irrigation ports and tracks arranged adjacent to said evacuation ports and tracks.

16. The device of claim 15 wherein said curved blade having a plurality of channels in a surface of a bottom component of said curved blade tip, a plurality of channels in a surface of a top component of said curved blade tip.

17. The device of claim 16 wherein said plurality of channels of said bottom component align with said evacuation ports, said plurality of channels of said top component align with said irrigation ports.

18. The device of claim 16 further comprising a membrane arranged between said bottom component and said top component of said curved blade tip.

19. The device of claim 13 further comprising a connector member arranged in a slot of a bottom component of said curved blade tip and a slot of the top component of said curved blade tip.

20. A method of retrieving diagnostic epithelial cells from the tonsillar crypts of both palatine or lingual tonsils with an endoscopic device, said method comprising the steps of:
selecting for the endoscopic device a conical tip if testing a palatine tonsil or a curved blade tip if testing a lingual tonsil;
securing one of said tips to a handle of the endoscopic device;
irrigating the tonsil with a saline solution via said tip of the endoscopic device;
evacuating a liquid tissue sample from the tonsil via said tip of the endoscopic device;
passing said lingual tonsil sample through a filter system;
removing the epithelial cells from said filter system for diagnostic purposes; and
removing any remaining waste of said liquid tissue sample to a waste container of the endoscopic device.
